(19) United States
(12) Patent Application Publication
(10) Pub. No.: US 2010/0129289 A1
(43) Pub. Date: May 27, 2010

(54) COMPOSITIONS AND METHODS FOR THE DETECTION OF VULNERABLE PLAQUE

(75) Inventors: Stephen C. Lee, Dublin, OH (US); Andrew D. Kebbel, Hamden, CT (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 12/301,417

(22) PCT Filed: May 18, 2007
(86) PCT No.: PCT/US07/69267

§ 371 Date: Feb. 3, 2010

Related U.S. Application Data
(60) Provisional application No. 60/747,654, filed on May 18, 2006.
(60) Provisional application No. 60/801,851, filed on May 19, 2006.

(51) Int. Cl. A61K 51/00; C07K 7/06; C07K 7/08; G01N 33/00; A61K 49/00; A61B 5/055; A61K 49/04

(52) U.S. Cl. 424/1.69; 530/328; 530/327; 530/329; 436/86; 424/9.1; 424/9.341; 424/9.4

(57) ABSTRACT

Targeting polypeptide imaging agents, methods employing the agents, and kits employing the agents are provided. The targeting polypeptide imaging agents may have at least one targeting polypeptide and at least one contrast enhancing imaging agent.

### CROSS REFERENCE TO RELATED APPLICATIONS

This application claims priority to U.S. Provisional Application Ser. No. 60/747,654, filed May 18, 2006, and U.S. Provisional Application Ser. No. 60/801,851, filed on May 19, 2006, the entireties of which are incorporated by reference herein.

### BACKGROUND

One of the primary health problems facing Americans today is cardiovascular disease. Cardiovascular disease results in more deaths every year than the next four leading causes of death combined (cancer, respiratory diseases, accidents, and diabetes). A particularly frightening fact is that about two thirds of unexpected cardiac deaths occur without manifestation of symptoms related to heart disease. Many of these unexpected deaths occur when a vulnerable plaque ruptures, exposing prothrombotic material to the circulation, often resulting in complete and catastrophic occlusion of the blood vessel. Thus, there remains a need in the art for improved methods and devices for detecting vulnerable plaque.

### SUMMARY

Disclosed herein are compositions and methods for the detection of vulnerable plaques. Compositions may comprise a targeting polypeptide imaging agent that may comprise at least one targeting polypeptide in spatial proximity to at least one contrast enhancing imaging agent. The targeting polypeptide imaging agent may have at least one targeting polypeptide covalently or non-covalently chemically bonded to at one contrast enhancing imaging agent. In some embodiments, the contrast enhancing imaging agent may be a paramagnetic contrast agent. The paramagnetic contrast agent may be chosen from gadolinium, cobalt, nickel, manganese, small particulate iron oxides, and ultra small particulate iron oxides, or combinations thereof. In some embodiments, the contrast enhancing imaging agent may comprise a contrast enhancing imaging agent selected from ¹³¹I, ¹²⁵I, ¹²³I, ^{99m}Tc, ¹⁸F, ⁶⁸Ga, ⁶⁷Ga, ⁷²As, ⁸⁹Zr, ⁶⁴Cu, ⁶²Cu, ¹¹¹In, ²⁰³Pb, ¹⁹⁸Hg, ¹¹C, ⁹⁷Ru, and ²⁰¹TI, or combinations thereof. In yet other embodiments, the contrast imaging agent may comprise a contrast-enhancing nanodevice.

Also disclosed herein are methods of detecting vulnerable plaque. In some embodiments, the methods may comprise the steps of supplying at least one targeting polypeptide imaging agent comprising at least one targeting polypeptide in spatial proximity to at least one contrast enhancing imaging agent such that the at least one targeting polypeptide imaging agent may come in contact with a target of interest, and subsequently applying an imaging technique to image the at least one targeting polypeptide imaging agent.

A kit comprising at least one targeting polypeptide imaging agent in combination with a pharmaceutically acceptable carrier, wherein the at least one targeting polypeptide imaging agent comprises at least one targeting polypeptide in spatial proximity to at least one contrast enhancing imaging agent is also contemplated.

### BRIEF DESCRIPTION OF THE SEVERAL VIEWS OF THE DRAWINGS

The following detailed description of embodiments of the present invention can be best understood when read in conjunction with the following drawings, where like structure is indicated with like reference numerals and in which:

FIG. 1 is a schematic of HAEC peptide binding assay. Lipid- and non-lipid-loaded human aortic endothelial cells were assayed to determine if the synthesized potential targeting peptides could bind human cells;

FIG. 2 is a schematic of resected tissue peptide binding assay. Peptides were tested on both WHHL and NZW tissues to test for binding to aortic intima;

FIG. 3 is a schematic of a pull-down experiment. The membrane proteins are isolated from WHHL rabbit aorta using the Mem-PER extraction kit from Pierce and are incubated with SA-coated iron oxide nanoparticles that are conjugated to biotinlyated peptide. The bound protein is separated magnetically, run on SDS-PAGE gel electrophoresis, and analyzed with mass spectroscopy;

FIG. 4 is a demonstration of successful lipid loading of HAECs. (a) Lipid-loaded HAECs after one week of growing in media containing 15 μg/mL acetylated LDL. The arrows indicate distinct accumulation of lipid. (b) Control cells without added lipid;

FIG. 5 illustrates the result of lipid-loaded HAEC assay for peptide 17. Absence of brown staining indicates negative result for binding. Image is 100× magnification;

FIG. 6 illustrates the result of lipid-loaded HAEC assay for peptide 23. Absence of brown staining indicates negative result for binding. Image is 100× magnification;

FIG. 7 illustrates the result of lipid-loaded HAEC assay for anti-FLAG and SA controls. Absence of brown staining indicates negative result for binding, which is a positive result for the case of the control experiments. (a) Result for anti-FLAG control, 200×. (b) Result for SA control, 200×;

FIG. 8 illustrates the result of WHHL assay with peptide 9. (a)-(d): Sections incubated with peptide 9 (1× concentration). (e)-(f): Anti-FLAG-HRP control sections;

FIG. 9 illustrates the result of WHHL assay with peptide 9. (a)-(d): Sections incubated with peptide 9 (1× concentration). (e)-(f): Anti-FLAG-HRP control sections.

FIG. 10 illustrates results of WHHL assay with peptides 3 and 4. (a)-(b): Sections incubated with peptide 3. (c)-(d): Sections incubated with peptide 4. (e)-(f): SA-HRP control sections;

FIG. 11 illustrates the result of WHHL assay with peptides 3 and 4. (a)-(b): Sections incubated with peptide 3. (c)-(d): Sections incubated with peptide 4. (e)-(f): SA-HRP control sections;

FIG. 12 illustrates the result of NZW assay with peptides 3, 4, and 9. (a), (c), (e): Sections incubated with peptide 3, peptide 4, and SA-HRP control, respectively. (b), (d), (f): Sections incubated with peptide 9 at 1× concentration (b) and (d), and anti-FLAG control.

FIG. 13 illustrates a comparison of 1× and 10× concentrations of peptide 9 on WHHL and NZW tissues. Top row: WHHL tissue. Bottom row: NZW tissue. (a)-(c): 1× concentration, 10× concentration, and anti-FLAG-HRP control on WHHL tissue. (d)-(f): 1× concentration, 10× concentration, and anti-FLAG-HRP control on NZW tissue;

FIG. 14 illustrates the result of WHHL assay with peptide 9R (randomized peptide 9 sequence). (a): Section incubated with peptide 9. (b): Section incubated with peptide 9R. (c): anti-FLAG-HRP control sections;

FIG. 15 illustrates the result of WHHL assay with peptides 3R and 4R (randomized peptide 3 and 4 sequences). (a)-(b): Sections incubated with peptide 3 and 3R, respectively. (c)-(d): Sections incubated with peptides 4 and 4R, respectively. (e): SA-HRP control sections;

FIG. 16 illustrates the binding of peptide 9 to WHHL aorta is localized to the intimal layer of the aorta. (a) The positive brown staining is clearly present only on the intimal surface with no infiltration into the plaque region. (b) Hoechst stain suggests presence of an intact endothelium along with positive brown staining of peptide 9 on the surface. Both images are 400× magnification;

FIG. 17 illustrates the binding of peptide 9 to WHHL aorta is seen over plaque-rich regions. (a) The positive brown staining is seen over intima covering plaque. (b) No stain is present over the intima covering a region with no plaque;

FIG. 18 illustrates the binding of peptides to WHHL aorta sections. (a)-(d): Peptides 1, 10, 20, and anti-FLAG-HRP control, respectively. (e)-(i): Peptides 6, 7, 11, 24, and SA-HRP control, respectively;

FIG. 19 illustrates the binding of peptides to WHHL aorta sections. (a)-(d): Peptides 2, 14, 16, and anti-FLAG-HRP control, respectively. (e)-(i): Peptides 6, 7, 12, 13, and SA-HRP control, respectively;

FIG. 20 illustrates the binding of peptides to WHHL aorta sections. (a)-(c): Peptides 24, 28, and SA-HRP control, respectively.

FIG. 21 illustrates the binding of peptides to WHHL aorta sections. (a)-(e): Peptides 34, 37, 38, 39, and SA-HRP control, respectively;

FIG. 22 illustrates the binding of peptide 9 to frozen WHHL aorta tissue sections. (a) Peptide 9 stains the intimal surface. (b) Peptide 9R shows no binding. (c) Anti-FLAG control. All images are 40× magnification;

FIG. 23 illustrates an experiment to determine location of nanoparticles on the NuPAGE gel. Four lanes (4-7) are shown flanked by standard ladders (3 and 8). Lanes 4 and 5 are the SA-coated nanoparticles conjugated to peptide 9 from heated and non-heated samples, respectively. Lanes 6 and 7 are SA-coated nanoparticles without conjugated peptide from heated and non-heated samples, respectively. (a) Result for loading buffer containing 10% glycerol without reducing agent. (b) Result for NuPAGE loading buffer containing reducing agent. Note that for the conjugated particles (lanes 4 and 5), no streak is observed on the gel using either system;

FIG. 24 illustrates the result of the initial pull-down experiment. Lanes 3 and 6 are the protein standard ladders. Lane 4 is the sample containing the isolate from the magnetic separation. Lane 5 is the sample containing the supernatant from the first magnetic separation step, which should contain several proteins; and

FIG. 25 illustrates the result of the attempt to improve membrane protein extraction efficiency. Lanes 1 and 9 are the protein standard ladders. Lane 4 is the sample containing the raw tissue homogenate without added reagents. Lane 5 is the sample containing the isolated membrane proteins from the Mem-PER extraction kit. The other lanes contain samples unrelated to this experiment. Approximate molecular weights are indicated on the figure.

### DETAILED DESCRIPTION OF EMBODIMENTS OF THE INVENTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

In accordance with embodiments of the invention, targeting polypeptide imaging agents comprising at least one targeting polypeptide and at least one contrast enhancing imaging agent are provided. The at least one targeting polypeptide and the at least one contrast enhancing imaging agent are in spatial proximity to one another. Spatial proximity between the targeting polypeptide and the contrast enhancing imaging agent may be effected in any manner which preserves the specificity of the targeting polypeptide for its target tissue. For example, spatial proximity between the contrast enhancing imaging agent and the targeting polypeptide may be effected by a covalent or non-covalent chemical bond. Such a chemical bond may be effected through a chelating substance and/or an auxiliary molecule, including, but not limited to, mannitol, gluconate, glucoheptonate, and tartrate. In other examples, such a chemical bond may be formed directly. Alternatively, spatial proximity between the contrast enhancing imaging agent and the targeting polypeptide may be effected by incorporating the contrast enhancing imaging agent and the targeting polypeptide in a micelle or liposome, in such a way that the affinity of the targeting polypeptide for its target tissue is maintained. Spatial proximity between the contrast enhancing imaging agent and the targeting polypeptide may also be effected by attaching the contrast enhancing imaging agent and the targeting polypeptide to a matrix such as a microsphere, liposome, or micelle. Thus, in some examples, the targeting polypeptide is conjugated to the contrast enhancing imaging agent. In other examples, the targeting polypeptide is not conjugated to the contrast enhancing imaging agent.

Any suitable targeting polypeptide may be used. The targeting polypeptide is selected to target at least one of plaque, vulnerable plaque, cardiovascular tissue, thrombi, diseased vasculature, or non-diseased vasculature. In some examples, the targeting polypeptide is selected to target vulnerable plaque. In some examples, the targeting polypeptide may have a sequence selected from: C-K-Q-S-F-E-K-S-C; S-S-L-P-A-P-P-W-P-L-R-G; T-S-P-Q-T-K-D-C; C-V-M-P-G-L-K-N-C; C-N-H-R-Y-M-Q-M-C; C-N-K-N-S-I-P-H-C; S-S-S-K-M-Q-A-A-H-Q-L-P; C-K-S-D-A-N-S-H-C; C-A-P-G-P-S-K-S-C; S-I-G-Y-P-L-P; C-K-Q-S-P-P-S-M-C; K-S-L-S-R-H-D-H-I-H-H-H; A-P-H-Y-L-K-T-A-P-P-P-N; C-H-P-A-S-S-P-Q-C; T-D-T-T-M-G-Q-V-H-R-H-P; N-A-D-N-Q-M-T-W-R-H-V-L; N-L-T-S-L-T-Q-G-S-A-M-L; T-P-L-E-V-H-P-E-S-L-P-W; Y-I-T-P-Y-A-H-L-R-G-G-N; and T-Q-T-P-I-K-H-H-L-L-K-E, or a sequence having at least 50%, 55%, 60%, 65%, 70%, 75%, 80%, 85%, 90%, 95%, 96%, 97%, 98%, 99%, 99.5%, or 99.9% identity or similarity to one of the targeting polypeptide sequences.

In some examples, the targeting polypeptide has a sequence of T-P-L-E-V-H-P-E-S-L-P-W. In other examples, the targeting polypeptide has a sequence of Y-I-T-P-Y-A-H-L-R-G-G-N. In yet further examples, the targeting polypeptide has a sequence of T-Q-T-P-I-K-H-H-L-L-K-E. In some examples, the targeting polypeptide sequence may have any suitable N- and/or C-terminal modifications. For example, the targeting polypeptide sequence may further comprise a C-terminal sequence comprising D-Y-K-D-D-D-K that may not contribute to recognition of vasculature but may be recognized by an antibody. This antibody provides a means to detect the peptide. In other examples, the targeting polypeptide sequence may further comprise at least one linker, such as a glycine linker, at the N-terminal or C-terminal. For example, G-G-G-S repeat units reiterated typically 1-5 times in the linker, polyproline linkers of any length, and/or a human IgG hinge region may be used as linkers. In yet further examples, the targeting polypeptide sequence may be N-terminal biotinylated. It will be understood that any suitable modifications may be made to the targeting polypeptide sequences that do not interfere with their targeting ability.

Any suitable contrast enhancing imaging agents may be used. For example, highly specific and sensitive contrast enhancing imaging agents are provided by radionuclides, which can then be detected, using positron emission tomography (PET) or Single Photon Emission Computed Tomography (SPECT) imaging. In some examples, the contrast enhancing imaging agents may contain a radionuclide selected from the group consisting of ¹³¹I, ¹²⁵I, ¹²³I, ^{99m}Tc, ¹⁸F, ⁶⁸Ga, ⁶⁷Ga, ⁷²As, ⁸⁹Zr, ⁶⁴Cu, ⁶²Cu, ²⁰³Pb, ¹⁹⁸Hg, ¹¹C, ⁹⁷Ru, and ²⁰¹TI or combinations thereof. In other examples, the contrast enhancing imaging agent may be a paramagnetic contrast agent, such as gadolinium, cobalt, nickel, manganese and iron. For example, the contrast enhancing imaging agent may be small particular iron oxides or ultras small particulate iron oxide. In yet further examples, the contrast enhancing imaging agent may be a contrast enhancing nanodevice. For example, the nanodevice discloses in Bryan R. Smith, Johannes Heverhagen, Michael Knopp, Petra Schmalbrock, John Shapiro, Masashi Shiomi, Nicanor Moldovan; Mauro Ferrari and Stephen C. Lee* (2007). Magnetic Resonance Imaging of atherosclerosis in vivo using biochemically targeted ultra-small superparamagnetic iron oxide particles (SPIONs). In press, Biomedical Microdevices, incorporated by reference herein.

In other examples, the contrast enhancing imaging agent may be more than one of the radionucleotides, paramagnetic contrast agents, and/or contrast enhancing nanodevices.

The contrast enhancing imaging agents may be incorporated into the targeting polypeptide imaging agent in any suitable manner. For example, as discussed above, the contrast enhancing imaging agent may be directly incorporated by covalent bonding directly to the targeting polypeptide, or the contrast enhancing imaging agent may be non-covalently or covalently associated with the targeting polypeptide through a chelating structure or through an auxiliary molecule, including, but not limited to, mannitol, gluconate, glucoheptonate, and tartrate. When a chelating structure is used to provide spatial proximity between the contrast enhancing imaging agent and the targeting polypeptide, the chelating structure may be directly associated with the targeting polypeptide or it may be associated with the targeting polypeptide through an auxiliary molecule, including, but not limited to, mannitol, gluconate, glucoheptonate, and tartrate.

Any suitable chelating structure may be used to provide spatial proximity between the contrast enhancing imaging agent and the targeting polypeptide of the agent through covalent or non-covalent association. Many such chelating structures are known in the art. For example, the chelating structure may be a N₂S₂ structure, an NS₃ structure, an N₄ structure, an isonitrile-containing structure, a hydrazine containing structure, a HYNIC (hydrazinonicotinic acid) group-containing structure, a 2-methylthiolnicotinic acid group-containing structure, or a carboxylate group containing structure, and combinations thereof. In some cases, chelation can be achieved without including a separate chelating structure, because the contrast enhancing imaging agent may chelate directly to the targeting polypeptide. It will be understood that any suitable methodology for associating the contrast enhancing imaging agent with the targeting polypeptide may be used.

In accordance with other embodiments of the invention, methods of detecting a target of interest are provided. The methods can comprise the steps of supplying at least one targeting polypeptide imaging agent comprising at least one targeting polypeptide in spatial proximity to at least one contrast enhancing imaging agent such that the at least one targeting polypeptide imaging agent may come in contact with a target of interest and subsequently applying an imaging technique to image the at least one targeting polypeptide imaging agent. The targeting polypeptide may bind to the target of interest and the contrast enhancing imaging agent may be imaged using any suitable imaging technique. Thus, the target of interest may be imaged using the methods.

It will be understood that the at least one targeting polypeptide imaging agent may be supplied in any suitable manner. For example, the at least one targeting polypeptide imaging agent may be supplied in vitro to a sample, such as a tissue sample, that may contain a target of interest. For example, the tissue sample may contain plaque, vulnerable plaque, cardiovascular tissue, or thrombi. In another example, the tissue sample may contain vulnerable plaque. In another example, the at least one targeting polypeptide imaging agent may be supplied in vivo to a subject of interest. Again, the targeting polypeptide imaging agent may be used to image a target of interest, including, but not limited to plaque, vulnerable plaque, cardiovascular tissue, or thrombi by applying a suitable imaging technique to the subject. In this manner, the targeting polypeptide imaging agent may be used to screen for or detect the presence of a target or targets of interest. It will be understood that the targeting polypeptide imaging agent may be administered in vitro or in vivo in any suitable manner. For example, a sample may be exposed to the targeting polypeptide imaging agent in vitro by washing the sample with the targeting polypeptide imaging agent in solution. In another example, a subject may be injected with the targeting polypeptide imaging agent.

Any suitable imaging technique may be utilized, and one having skill in the art will be able to select an imaging technique that may be used to image a particular targeting polypeptide imaging agent. For example, the imaging technique may be selected from computed tomography scanning, computerized axial tomography scanning, positron emission tomography scanning, gamma detection, and magnetic resonance imaging or combinations thereof

In some examples, the targeting polypeptide imaging agents of the invention may be used in accordance with the methods of the invention by those of skill in the art to image plaque in the cardiovascular system of a subject. Images may be generated by virtue of differences in the spatial distribution of the imaging agents which accumulate in the various tissues and organs of the subject. The spatial distribution of the imaging agent accumulated may be measured using any suitable means. Some cardiovascular lesions may be evident when a less intense spot appears within the image, indicating the presence of tissue in which a lower concentration of imaging agent accumulates relative to the concentration of imaging agent which accumulates in surrounding cardiovascular tissue. Alternatively, a cardiovascular lesion might be detectable as a more intense spot within the image, indicating a region of enhanced concentration of the imaging agent at the site of the lesion relative to the concentration of agent which accumulates in surrounding cardiovascular tissue. Accumulation of lower or higher amounts of the imaging at the site of a lesion may readily be detected visually, by inspection of the image of the cardiovascular tissue. Alternatively, the extent of accumulation of the imaging agent may be quantified using known methods for quantifying radioactive emissions. In some examples, more than one imaging agent may be used to perform simultaneous studies.

An effective amount of at least one targeting polypeptide imaging agent may be combined with a pharmaceutically acceptable carrier for use in imaging studies. In accordance with the invention, “an effective amount” of the imaging agent of the invention is defined as an amount sufficient to yield an acceptable image using the available equipment. In some examples, an effective amount of the imaging agent of the invention may be administered in more than one injection. Effective amounts of the imaging agent of the invention will vary according to factors such as the degree of susceptibility of the individual, the age, sex, and weight of the individual, idiosyncratic responses of the individual, the dosimetry. Effective amounts of the imaging agent of the invention will also vary according to instrument and film-related factors. Optimization of such factors is within the level of skill in the art. In some examples, the effective amount may be in the range of from about 0.1 to about 10 mg by injection or from about 5 to about 100 mg orally for use with MRI.

In some examples, the at least one targeting polypeptide imaging agent can be administered to a subject in accordance with any means that facilitates accumulation of the agent in a subject's cardiovascular system. For example, the imaging agent of the invention may be administered by arterial or venous injection, and may be formulated as a sterile, pyrogen-free, parenterally acceptable aqueous solution. The preparation of such parenterally acceptable solutions, having due regard to pH, isotonicity, stability, and the like, is within the skill in the art. In some examples, a formulation for intravenous injection may contain, in addition to the targeting polypeptide imaging agent, an isotonic vehicle such as Sodium Chloride Injection, Ringer's Injection, Dextrose Injection, Dextrose and Sodium Chloride Injection, Lactated Ringer's Injection, or any other suitable vehicle.

The amount of imaging agent used for diagnostic purposes and the duration of the imaging study will depend upon the nature and severity of the condition being treated, on the nature of therapeutic treatments which the patient has undergone, and on the idiosyncratic responses of the patient. Ultimately, the attending physician will decide the amount of imaging agent to administer to each individual patient and the duration of the imaging study.

In yet further embodiments, kits for imaging a desired target are provided. The kits may comprise at least one of the targeting polypeptide imaging agents in combination with a pharmaceutically acceptable carrier. Any suitable pharmaceutically acceptable carrier may be used. For example, the pharmaceutically acceptable carrier may comprise human serum albumin. Human serum albumin may be made in any way, for example, through purification of the protein from human serum or though recombinant expression of a vector containing a gene encoding human serum albumin. Other suitable carriers include, but are not limited to, detergents, dilute alcohols, carbohydrates, and auxiliary molecules or combinations thereof. Yet further suitable carriers include, but are not limited to, any and all solvents, dispersion media, coatings, antibacterial and antifungal agents, isotonic agents, absorption delaying agents, and the like

The formulation used in the present invention may also contain stabilizers, preservatives, buffers, antioxidants, or other additives known to those of skill in the art. The use of such media and agents for pharmaceutically active substances is well known in the art. Supplementary active compounds can also be incorporated into the imaging agent or the invention. The imaging agent of the invention may further be administered to an individual in an appropriate diluent or adjuvant, co-administered with enzyme inhibitors or in an appropriate carrier such as human serum albumin or liposomes. Pharmaceutically acceptable diluents include sterile saline and other aqueous buffer solutions. Adjuvants contemplated herein include resorcinols, non-ionic surfactants such as polyoxyethylene oleyl ether and n-hexadecyl polyethylene ether. Enzyme inhibitors include pancreatic trypsin inhibitor, diethylpyrocarbonate, and trasylol. Liposomes inhibitors include water-in-oil-in-water CGF emulsions as well as conventional liposomes (Strejan et al., J Neuroimmunol 7:27 [1984]). The kit may further comprise additional items to facilitate the use of the kit. For example, the kit may comprise syringes, instructions, and reaction vials or combinations thereof

Any suitable amount of the at least one targeting polypeptide imaging agent and the pharmaceutically acceptable carrier may be used. For example, the kit may contain from about 1 to about 30 mCi of the at least one targeting polypeptide imaging agent described above, in combination with a pharmaceutically acceptable carrier. The targeting polypeptide imaging agent and carrier may be provided in any suitable form. For example, the targeting polypeptide imaging agent and carrier may be provided in solution or in lyophilized form. When the targeting polypeptide imaging agent and carrier of the kit are in lyophilized form, the kit may optionally contain a sterile and physiologically acceptable reconstitution medium such as water, saline, buffered saline, and the like.

### Examples

Methods and Materials

Phage Display Libraries

Candidates for peptides that target plaque were all discovered using two commercially available phage display peptide library kits (New England Biolabs, MA). The Ph.D.-12 kit consists of 12-mer amino acid sequences and contains approximately 1.9×10⁹ independent clones. The second kit is the Ph.D.-C7C, which is comprised of 1.2×10⁹ 7-mer sequences that are flanked on both sides by cysteine residues, thus producing 9-mers of the form Cys-(7-mer)-Cys. Under non-reducing conditions the cysteines spontaneously form a disulfide cross-link, which results in phage display of cyclized peptides, in contrast to the linear 12-mers.

Experimental Animal Model of Atherosclerosis

One of the animal models is the Watanabe heritable hyperlipidemic (WHHL) rabbit. The WHHL model is genetically predisposed toward the formation of atherosclerotic plaques [38], and the animals were purchased from either Covance (Princeton, N.J.) or Brown Family Enterprises/Gemini Research (Odenville, Ala.). The WHHL rabbit develops fairly occlusive plaque in the aorta increasingly with age. The disease seen in this strain is quite similar to the human condition of familial hyperlipidemia, as demonstrated by the fact that the lipoprotein type found in the vascular lesions is nearly identical. The WHHL rabbit develops not only atherosclerosis, but also xanthomas (fat deposits under the surface of the skin), fatty infiltration of organs, and elevated lipids on a normal diet. Usually, severe coronary artery disease occurs by age two in these animals. While this animal model is generally accepted, there are some important differences between the disease progression in humans and WHHL rabbits. Primarily, the animals' cholesterol metabolism is fundamentally different, because rabbits are herbivores. In addition to this, the primary location of atherosclerosis in man is the abdominal aorta, but develops in the aortic arch and thoracic aorta in the WHHL model. Nonetheless, the WHHL rabbit model provides an easily accessible source of plaque-laden vasculature that can be used to isolate targeting agents for atherosclerotic plaque.

Recently, a spontaneous myocardial infarction (MI) version of the model, the WHHLMI, has been developed [39], and is used in some of the experiments as well (Masahashi Shiomi, Kobe, Japan). WHHLMI rabbits experience spontaneous lethal vascular events and have plaques with similar morphology to the vulnerable plaques seen in humans. Furthermore, the plaques in the WHHLMI model often exhibit evidence of prior rupture, which is the case in humans as well [40]. In spite of this improvement with respect to modeling the human disease, the WHHLMI is still not a perfect model. It has been shown that in most cases, vulnerable plaque rupture is not the primary cause of the fatal coronary events experienced by most of the WHHLMI rabbits; additional triggering factors are required to promote plaque rupture and thrombogenesis. However, the animal models are a good compromise between disease progression, cost, and accessibility.

The control animal for the WHHL/MI rabbit models is the New Zealand white (NZW) rabbit. This animal rarely shows evidence of large lesions, and will typically only develop plaque when fed a high cholesterol diet. Like the WHHL animals, the NZW rabbits are supplied by Covance (Princeton, N.J.). All animal studies were approved by The Ohio State University Institutional Laboratory Animal Care and Use Committee and performed in compliance with accepted standards of laboratory animal care.

Isolation of Peptides that Bind Plaque: Biopanning

To isolate peptides that bind specifically to the diseased intima in the atherosclerotic lesions of the WHHL rabbits, both the Ph.D.-12 and -C7C libraries were administered simultaneously in the ear vein of the animal. The phage were suspended in a small volume of Hanks buffered salt solution and circulated for 30 minutes in the anesthetized animal. Following the incubation with the phage libraries, the animal was perfused through the left ventricle with 200-300 mL of Hanks buffered salt solution to wash away nonspecifically bound phage in the aorta. The aorta was then removed, and the bound phage were eluted according to the protocol provided by New England Biolabs. The eluted phage were amplified in the recommended host bacteria, purified, and injected into another WHHL rabbit. This process was repeated in four rabbits, and the sequences of 400 isolated peptides were determined from the DNA sequences of the individual phage clones.

Isolation of Peptides that Bind Lipid-Loaded HAECs: Panning

Human aortic endothelial cells were purchased from Cambrex (CC2535) and cultured in the associated media according to the recommendations of the company. Cells were grown to confluency after which time acetylated low-density lipoprotein from a human source (Invitrogen, L35354) was added to the media at a concentration of 15 μg/mL. Cells were fed lipid-loaded media every three days for a total of three feedings. Panning with the phage libraries was conducted as previously described and was concluded after three rounds.

Selection of Targeting Peptides

The isolated peptides from the in vivo biopanning on WHHL rabbits and the in vitro panning on lipid-loaded HAECs were analyzed to determine if any homology was present throughout the different rounds. Several sequences were selected based on this criterion that show consensus sequences 3-5 amino acids in length. Another criterion for selection was if individual peptides were identified repeatedly during the various rounds of biopanning on WHHL rabbits. BLAST searches were also performed on all the isolated peptides to identify peptide sequences that are present in proteins associated with atherosclerosis. Peptides with 6/7 or 9/12 conserved sequences, depending on the length of the amino acid sequence, were considered for potential targeting agents. More than 40 individual peptide sequences were selected and synthesized by GenScript Corporation (Piscataway, N.J.) with >85% purity for all peptides. The peptides were synthesized with either an N-terminal biotin or a C-terminal FLAG sequence, DYKDDDDK, for the detection assays.

Description of Peptide Assays

Several experimental methods were employed to validate the binding capabilities of the synthesized peptides. The following discussions describe the assays.

Lipid-Loaded HAECs

Human aortic endothelial cells (HAECs) were loaded with acetylated low-density lipoprotein as described herein. Peptide binding assays were performed 24 hours after the third lipid-loading of the cells. Control cells grown in the standard media without added lipid were cultured for the same amount of time as the lipid-loaded HAECs.

The general procedure for the peptide binding assays was conducted as follows (see FIG. 1). Variations on this method will be discussed as well. Both the lipid- and non-lipid-loaded cells were imaged prior to the assays to verify morphological differences between the two conditions of the cells. The media was aspirated and cells were washed 3× with cold wash buffer (PBS w/Ca²⁺, Mg²⁻ with 2% FBS), followed by a 5 minute incubation on ice to minimize internalization of peptides. Cold blocking buffer (PBS w/1% BSA) was added to the cells for 15 minutes to prevent nonspecific binding. Peptides were then added to the cells in cold blocking buffer at 1, 5, or 100 μg/mL and incubated for 1 hour on ice. Following incubation, cells were washed 3×, and either streptavidin horseradish peroxidase (SA-HRP) or anti-FLAG horseradish peroxidase (anti-FLAG-HRP) was added to the cells in cold blocking buffer at the recommended concentrations provided by the manufacturers. Anti-FLAG monoclonal m2 antibody-peroxidase conjugate was purchased from Sigma (A8592), as well as SA-HRP (S2438). After a 1 hour incubation, cells were washed 5× and were incubated with diaminobenzidine (DAB, Sigma, D5905) for 30 minutes (1 DAB tablet, 15 mL wash buffer, 12 μL 30% H₂O₂, followed by which results in a brown colored precipitate in the presence of HRP. Following the incubation with DAB, cells were washed 3× and fixed with 10% buffered formalin for 10 minutes. Following a final wash to remove the formalin, cells were imaged to determine if brown staining was present. SA-HRP and anti-FLAG-HRP were added directly to cells that had not been incubated with peptide as a control to determine if the presence of brown truly indicated positive peptide binding.

Several variations on this general assay were performed to find an optimal assay system. As mentioned previously, peptide concentrations ranged from 1 to 100 μg/mL throughout the experiments. In one variation of the assay, peptides were added directly to the media, bypassing the washing and blocking steps. In this case the cells were incubated at 37° C., 5% CO₂ for 1-2 hours. This technique offered a greater chance that the peptides could be internalized by the cells compared to the cold version described above, so it was necessary to permeabilize the cells before adding SA-HRP or anti-FLAG-HRP. This was performed by adding either a 0.5% Triton-X solution for 10 minutes followed by a 20 minute incubation with 0.03% hydrogen peroxide, or by first fixing the cells with 4% paraformaldehyde for 15 minutes followed by permeabilization with cold methanol for 10 minutes.

The composition of the blocking buffer was also varied throughout the experiments. Initially, the blocking buffer contained 2% FBS, 1% BSA, 0.1% cold fish gelatin, and 0.05% sodium azide. Other compositions were 2% BSA, 1% BSA, and 0.5% BSA. The effect of counterstaining with hemotoxylin was also tested in some experiments either before or after fixing the cells to observe if brown staining was easier to detect on stained cells. The controls for this experiment were comparing the binding of each peptide to lipid- and non-lipid-loaded cells, and two wells of cells were incubated with only blocking buffer (no peptide) to test for binding of the SA-HRP and anti-FLAG-HRP to the cells.

Resected WHHL and NZW Rabbit Tissue

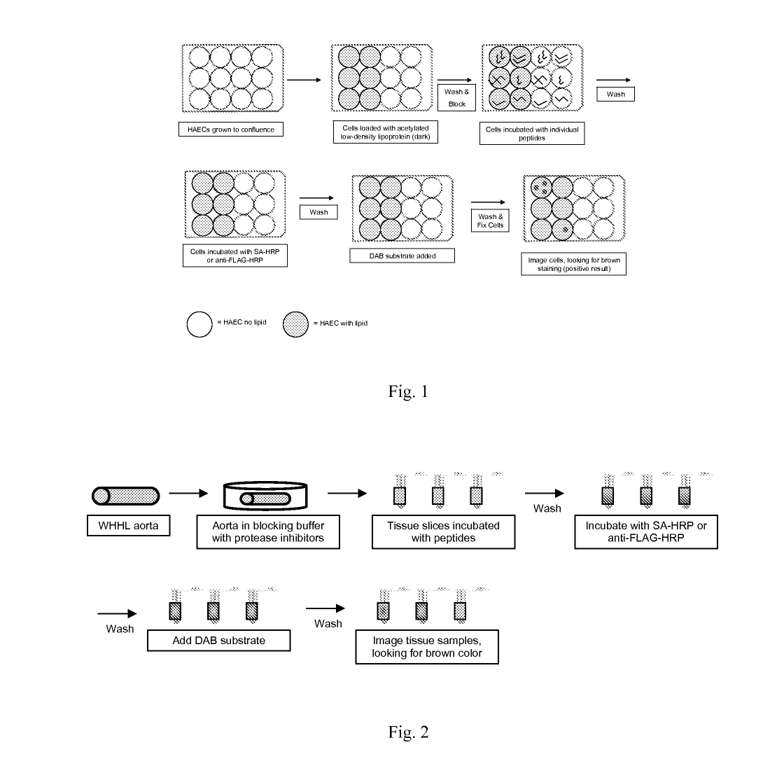

Experiments were performed to determine if the synthesized peptides could bind to the intima of the WHHL (diseased) rabbits but not to the NZW (control) rabbits. The following procedure is shown schematically in FIG. 2. The animals were anesthetized, injected with 0.7-1.0 mL heparin, and perfused with 200-300 mL Hanks buffered salt solution containing 1% BSA. A segment of the aorta comprising the aortic arch to the bifurcation of the aorta in the lower abdomen was removed and placed in a buffer solution of Hanks w/1% BSA and 1% protease inhibitor cocktail (Sigma, P8340). The aorta segments were opened longitudinally and sliced into individual pieces approximately 1-2 mm² each. These pieces were then placed into tubes containing individual peptides at a concentration of 5 μg/mL in the same buffer solution described above.

The tissue sections were incubated with individual peptides for 45-60 minutes at room temperature. Samples were washed 3× with PBS followed by incubation with SA-HRP or anti-FLAG-HRP in the same buffer for 45-60 minutes at room temperature. After another 3× washing, the samples were incubated with DAB for 30 minutes in the same buffer. After a final round of washing, tissue samples were imaged with a dissecting microscope and analyzed for the presence of brown staining on the endothelial surface. Tissue sections that showed significant staining were fixed with 10% buffered formalin and sectioned for further analysis to determine the location of the staining (e.g., endothelium, smooth muscle). Some tissue sections received no peptide incubation; rather they were exposed only to SA-HRP or anti-FLAG-HRP as a control.

Direct Staining on Frozen WHHL and NZW Rabbit Tissue

Some of the resected WHHL and NZW aortic arch tissues were frozen and sectioned into 4 μm slices. Binding assays were performed on these frozen sections in a similar manner to that described herein. Sections were thawed and incubated with blocking buffer (Hanks w/1% BSA) for 30 minutes. Individual peptides were added to the slides in blocking buffer at a concentration of 1 μg/mL. After 45 minutes of incubation, the slides were washed and either SA-HRP or anti-FLAG-HRP, depending on the modification of the peptides, was added at the recommended concentration and allowed to incubate for 1 hour. The slides were washed again, and the DAB substrate was added and incubated for 30 minutes while looking for brown color development. After washing away the DAB, the tissue samples were fixed with 10% buffered formalin for 10 minutes followed by counterstaining with hemotoxylin. The slides were then mounted with permount and glass coverslips for imaging. The standard SA-HRP and anti-FLAG-HRP controls were performed for these experiments as well.

ApoE Knockout Mice

The ApoE glycoprotein is involved with cholesterol transport activity among cells, and the knockout animal model exhibits five times the normal serum plasma cholesterol level as the control model and develops spontaneous atherosclerotic lesions that are similar in morphology to the human disease [41]. In the peptide binding assays, individual peptides are injected into an anesthetized mouse and allowed to circulate in vivo for 1 hour. Note that this is different from the WHHL/NZW binding assays where the aorta is first resected and then incubated with peptide. The animal is then fixed via cardiac perfusion with 4% formaldehyde, and the aorta is removed to test for binding of the peptide. The detection assay is the same assay described herein: either SA-HRP or anti-FLAG-HRP is added to the solution containing the resected tissue, followed by the addition of the DAB substrate, after which the tissue samples are sectioned and imaged.

Pull-Down Experiment

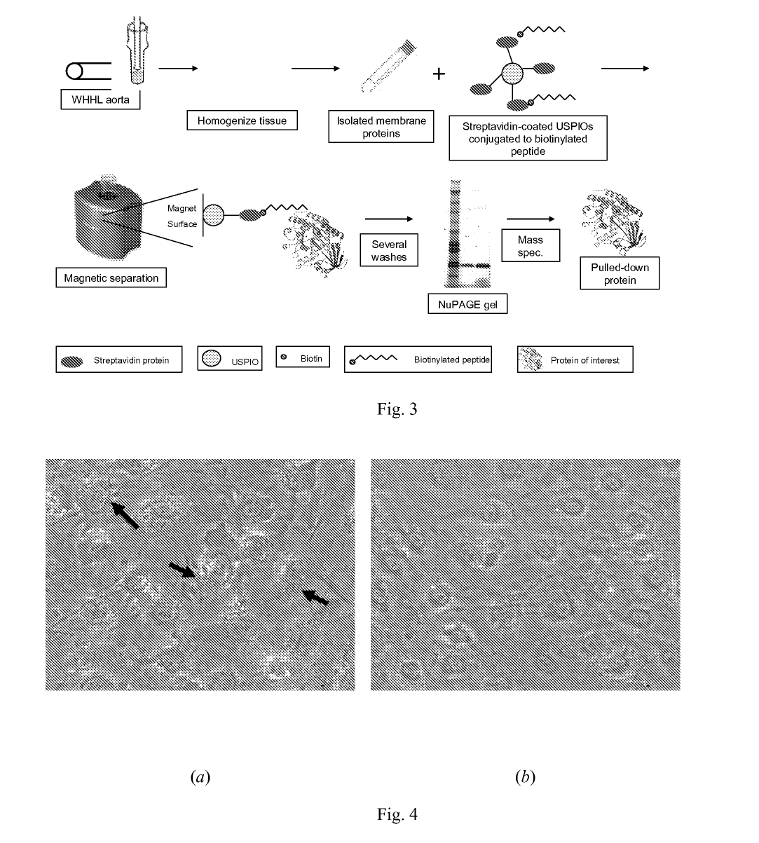

An initial attempt was made to isolate the protein to which the targeting peptides bind in vivo using a pull-down type experiment. The technique utilized to “pull down” the protein of interest is a magnetic separation. Iron oxide nanoparticles coated with streptavidin (Miltenyi Biotech, Germany) are washed to remove the azide from the buffer solution and are incubated with biotinylated peptide 9 to create a targeting agent for the protein that can be isolated via magnetic separation (see FIG. 3). The experiment begins by resecting a portion of WHHL aorta and homogenizing the tissue sample in a glass dounce (Kontes, 885300-0002). The Mem-PER Eukaryotic Membrane Protein Extraction Kit (Pierce Biotechnology, 89826) is used to isolate the hydrophobic membrane proteins where it is assumed the protein to which the targeting peptide binds may be found. The SA-coated iron oxide nanoparticles conjugated to the biotinylated peptide are added to the isolated membrane protein solution. The peptide concentration is approximately 15 μg/mL. The mixture is incubated for 2 hours at 4° C. and is then placed in a magnetic separator and left overnight at 4° C. to separate the bound protein from the bulk proteins.

After the initial overnight separation, the supernatant is collected and the sample is washed with PBS containing protease inhibitors (Sigma, P8340). The sample is incubated in the magnetic separator at 4° C. for 3-4 hours followed by a second wash step and another 3-4 hour separation. After the final separation the supernatant is poured off and the particles are centrifuged at 1000 rpm for 2 minutes in order to resuspend the particles in the remaining buffer. This avoids diluting the protein concentration by adding more buffer. The sample is then prepared for gel electrophoresis.

The NuPAGE Novex gel system from Invitrogen is used for protein separation. The 4-12% Bis-Tris gel (NP0321BOX) is combined with the MES SDS running buffer (NP0002) to create a system with a large protein size separation range (2.5-200 kDa). The sample buffer is the NuPAGE LDS sample buffer (NP0007), and all samples are prepared with sample reducing agent (NP0004) and run with antioxidant (NP0005) in the running buffer to avoid re-oxidation during the electrophoresis. Samples are prepared according to the NuPAGE system manual, and the gel is run using the XCe11 SureLock (EI0001) mini-cell system. A protein standard (SeeBlue Plus2, LC5925) is run on the gel for size estimation. After the electrophoresis the gel is stained with Coomassie Blue (SimplyBlue SafeStain, LC6060) to visualize protein bands. Bands of interest are then excised for mass spectrometry analysis.

### Example 1

### Biopanning Results

In vivo biopanning was performed on the WHHL rabbit animal model as described herein using both the 12-mer linear peptide and 7-mer disulfide bond constrained peptide phage display libraries. Panning was also performed on lipid-loaded HAECs as described herein using both libraries as well. From the panning procedures over 200 novel peptides were discovered de novo that are potential targeting agents for atherosclerotic plaque. Forty five of these candidates were selected to be synthesized for further binding assays based on specific selection criteria. These peptides are shown in Table 1. The peptides were modified by either N-terminal biotinylation or C-terminal FLAG sequence, DYKDDDDK. The biotinlyated peptides are assayed for binding using SA-HRP, while the FLAG sequence is detected by anti-FLAG-HRP.

TABLE 1
Peptides selected for further binding assays.

| | | | C-terminal |
|---|---|---|---|
| Peptide | | N-terminal | FLAG |
| Number | Sequence | Biotinylation | (DYKDDDDK) |
| 1 | CKQSFEKSC | | 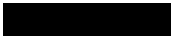 |
| 2 | CKSDANSHC | | 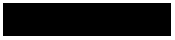 |
| 3 | TPLEVHPESLPW | 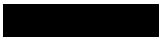 | |
| 4 | YITPYAHLRGGN | 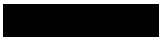 | |
| 5 | TPLTPNGLTRSG | | 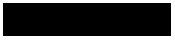 |
| 6 | SSLPAPPWPLRG | 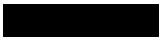 | |
| 7 | TSPQTKDC | 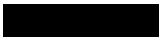 | |
| 8 | CSFNNRGIC | 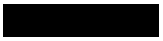 | |
| 9 | TQTPIKHHLLKE | | 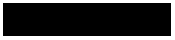 |
| 10 | CVMPGLKNC | | 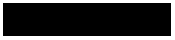 |
| 11 | CNHRYMQMC | 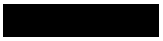 | |
| 12 | CAPGPSKSC | 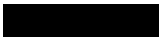 | |
| 13 | SIGYPLP | 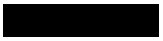 | |
| 14 | CKQSPPSMC | | 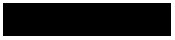 |
| 15 | SHHIPSYQWPLH | | 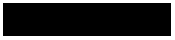 |
| 16 | KSLSRHDHIHHH | | 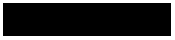 |
| 17 | TAPHHSTPANVP | | 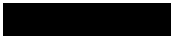 |
| 18 | KLPVNSTAPTST | | 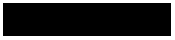 |
| 19 | CSWTQRNVC | | 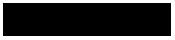 |
| 20 | CNKNSIPHC | | 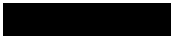 |
| 21 | CTGPSNESC | | 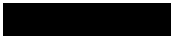 |
| 22 | CLKSAGLTC | | 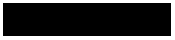 |
| 23 | CDSIKWPNC | | 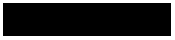 |
| 24 | SSSKMQAAHQLP | 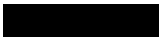 | |
| 25 | CTPTYPVRC | 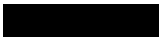 | |
| 26 | QANIPPPSASGH | 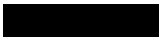 | |
| 27 | CLPSSTKQC | 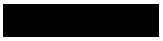 | |
| 28 | APHYLKTAPPPN | 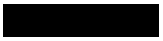 | |
| 29 | IVKPGNDGMARS | 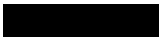 | |
| 30 | TQRIPWQPVGIS | 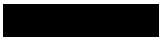 | |
| 31 | LLADTTHHRPWT | 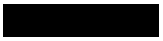 | |
| 32 | SPFSINEFLLPK | 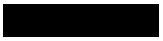 | |
| 33 | NLFQYPSFTAGT | 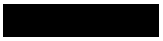 | |
| 34 | CHPASSPQC | 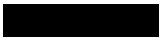 | |
| 35 | CQGRTSLTC | 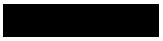 | |
| 36 | CNKIQDQWC | 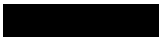 | |
| 37 | TDTTMGQVHRHP | 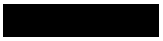 | |
| 38 | NADNQMTWRHVL | 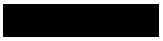 | |
| 39 | NLTSLTQGSAML | 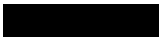 | |
| 40 | NISPILHTRLGT | 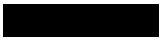 | |
| 41 | APQLPWSARPAV | 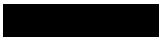 | |
| 42 | SPYPSWSTPAGR | 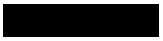 | |
| 43 | ILTHEHTLPK | | 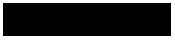 |
| 44 | PPEEWSPVTHLL | 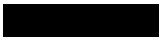 | |
| 45 | RNPGYTYHIALG | 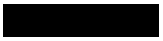 | |

The 45 peptides listed above were selected from the biopanning on WHHL rabbits and the panning on lipid-loaded HAECs. The peptides were modified by either N-terminal biotinylation or C-terminal FLAG sequence, D-Y-K-D-D-D-D-K, for the detection assays. The specific modification for each peptide is indicated by the shaded box.

A factor in selecting a peptide was whether it showed homology to other peptides isolated from individual rounds of panning For example, the peptide C(KQSFEKS)C was selected based on its homology to the sequences KQSPPSM and KQSQWHS, where the flanking cysteine residues are shown for only the first peptide for simplicity; the reader should be aware that 7-mers described in this discussion are all flanked by cysteine residues. The fact that certain peptides showed distinct homology to others isolated from the panning experiments may not necessarily develop a criterion for which of the homologous peptides to select. Therefore, there existed some arbitrary selection based on the homologous criterion. Another major selection criterion was if a particular peptide sequence appeared throughout multiple rounds of the panning experiments. An example of this is the 12-mer, TPLEVHPESLPW, which appeared in two rounds of biopanning Furthermore, this sequence shows some homology to another isolated 12-mer, TPLTPNGLTRSG, which increased the potential for this peptide to be a good targeting agent. The final selection criterion was the use of a BLAST search. Some peptides showed homology to known proteins associated with atherosclerosis or inflammatory processes in general. An example of this homology is the 12-mer, TPLTPNGLTRSG, which was mentioned above as being homologous to a 12-mer that appeared in two rounds of biopanning. This peptide shows good homology to a LDL receptor related protein (conserved sequence: LTPNGLT), further indicating the potential for this peptide to target plaque.

Three peptides were synthesized that were not selected from the biopanning on WHHL rabbits or the panning on lipid-loaded HAECs. These peptides have the following sequences: CNHRYMQMC (peptide 11), CAPGPSKSC (peptide 12), and SIGYPLP (peptide 13). The first peptide, CNHRYMQMC, was shown to bind to the atherosclerotic lesion endothelium of ApoE knockout mice in vivo, while the second peptide, CAPGPSKSC, bound to both ApoE knockout mice in vivo and also human atherosclerotic lesions ex vivo.

### Example 2

### Lipid-Loaded HAECs Peptide Binding Results

As described above, human aortic endothelial cells (HAECs) were loaded with media containing LDL, which mimics an early process in atherosclerotic plaque development. HAECs without added LDL were simultaneously cultured to provide a control for the lipid-loaded cells. To demonstrate uptake of lipid by the HAECs, images of both cell conditions were acquired, and the results are displayed in FIG. 4. The image shown in (a) shows the HAECs after one week of lipid-loading, while the image shown in (b) shows the control cells without added lipid. The arrows in (a) indicate regions of accumulated lipid. Note that no such morphological areas are seen in (b). Both images are 400× magnification. The difference in the photographic quality of the images is due solely to the software used to acquire the images (SPOT).

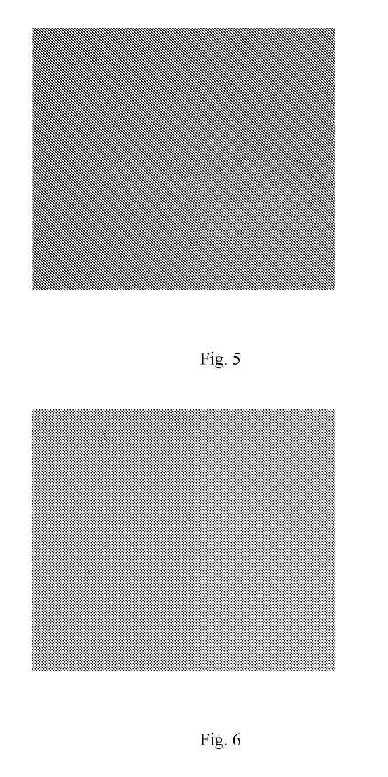

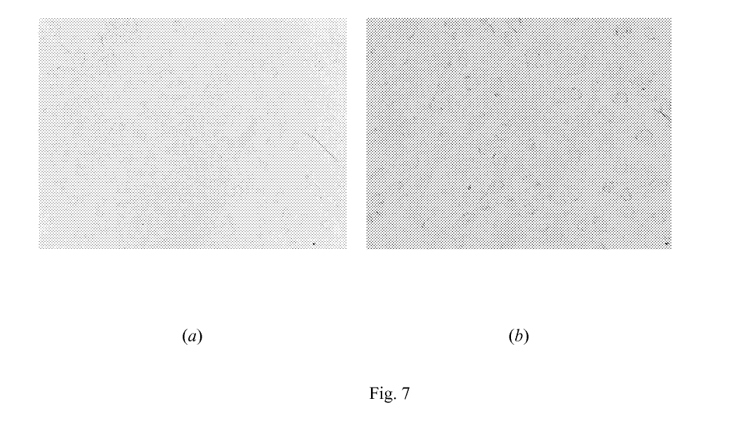

The binding assay on the lipid-loaded HAEC system was performed for peptides 1-33, excluding peptide 8 (see Table 1 for peptide listing). The experiments indicate that no positive binding was observed for any of the peptides that were assayed on the lipid-loaded HAECs. A representative sample of results is shown in FIGS. 5 and 6. Both images show no brown staining, which indicates a negative result for binding. FIG. 7 shows images from the SA-HRP and anti-FLAG-HRP controls. The control experiments show negative results for binding, which indicates that the detection antibody or protein does not bind to HAECs itself.

### Example 3

### Resected Aortic Tissue Binding Results

All peptides in Table 1 were assayed for binding to both WHHL and NZW rabbit aortic tissue as described above. The most generalized statement about the results of the peptides binding to the intimal surface of the WHHL and NZW rabbit aortas is that the binding is widely variable except in the case of peptide 9. Peptide 9 consistently shows staining on the WHHL aortic tissue. Furthermore, peptide 9 binds regions of plaque in a dose-dependent manner. Peptide 9 also shows some slight staining on the NZW tissue; however, this staining is shown to represent a consistent background staining, because the overall staining does not increase with an increase in the peptide concentration. Other peptides that show above-average reproducible staining on WHHL tissue are peptides 3 and 4. The figures that follow will show the results for these three peptides of interest.

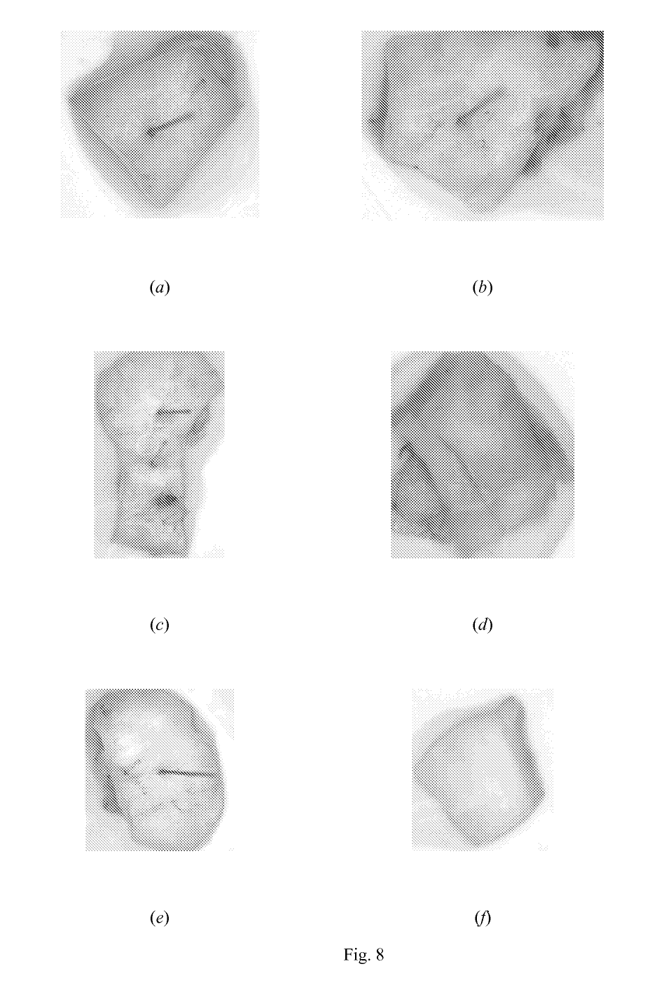

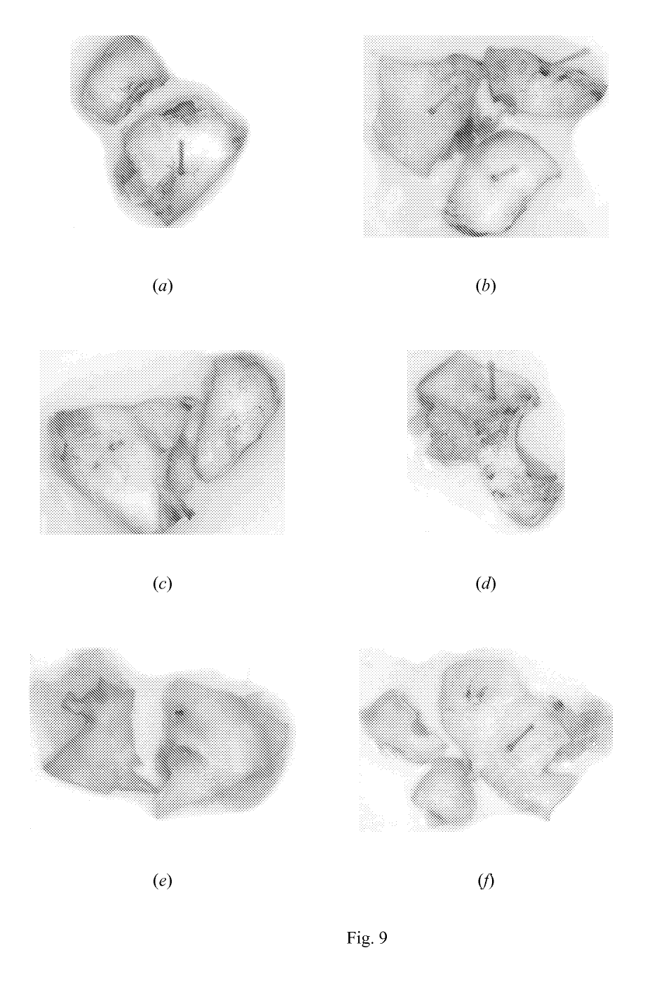

FIGS. 8 and 9 show the results of the binding assay for peptide 9 (C-terminal FLAG sequence) from two experiments performed on aortic tissue samples from two distinct WHHL rabbits. The sections shown in (a)-(d) were incubated with a 1× concentration of peptide 9 (˜10 μg/mL) and assayed according to Section 2.6.2, while those shown in (e)-(f) are the control sections that were not incubated with peptide and were used to determine whether anti-FLAG-HRP bound to the tissue itself. Note that the controls in FIGS. 8(e) and 9(f) show some regions of brown (positive) staining, but the other control sections are clear. Relative to the staining seen in the control sections, the sections showing staining in (a)-(d) in both figures show more total staining, and the staining is very distinct. There are some tissue sections that were incubated with peptide 9 that did not show staining. The fact that the tissue is heterogeneous could account for this observation.

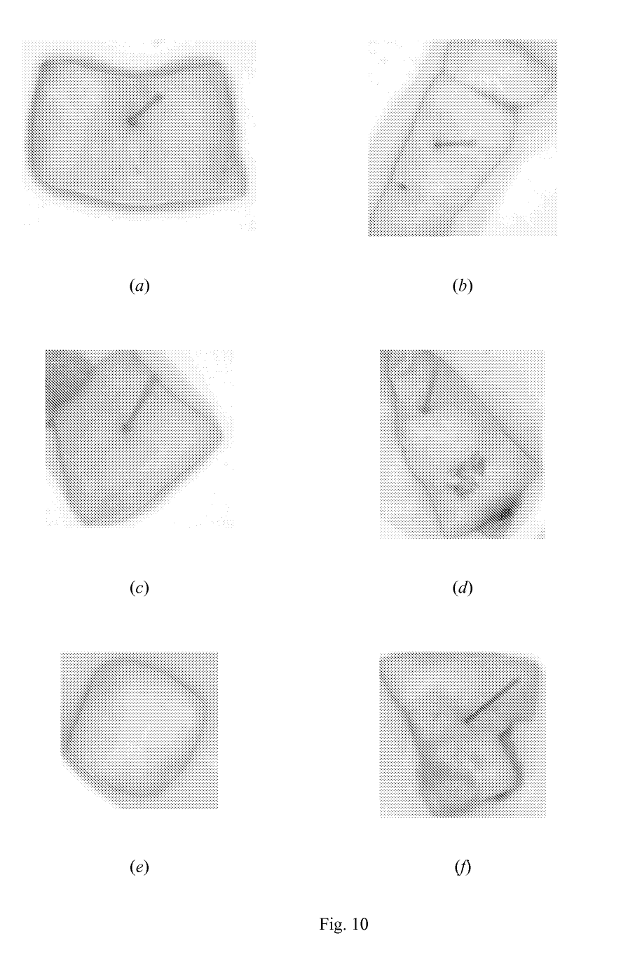

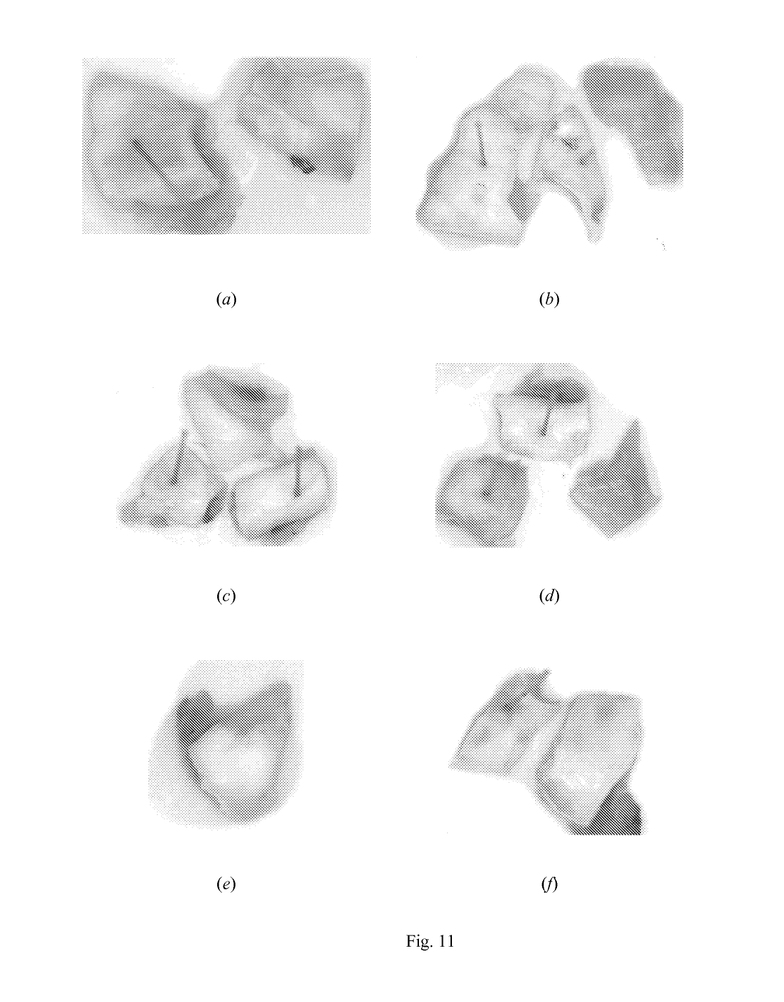

FIGS. 10 and 11 show the results of the binding assay for peptides 3 and 4 (both N-terminal biotinylation) performed on sections from the same WHHL rabbits as in FIGS. 7 and 8, respectively. The sections shown in (a) and (b) were incubated with peptide 3, and those shown in (c) and (d) were incubated with peptide 4. The sections were assayed according to the methods above. The sections shown in (e)-(f) are the control sections that were not incubated with peptide and were used to determine whether SA-HRP bound to the tissue itself. Note that the controls for the biotinylated peptides, (e)-(f), show much less staining than the control for peptide 9, which is detected by anti-FLAG-HRP. Peptide 4 shows good staining in FIG. 10(d), whereas peptide 3 yields the best staining in FIG. 11(b). However, the corresponding tissue samples in FIG. 10(c) for peptide 4 and FIG. 11(a) for peptide 3 show very little, if any, brown staining. This demonstrates the variability of peptide binding not only between different rabbits, but also the variability within each animal. This variability between different animals was present throughout the experiments for all of the peptides that showed binding activity, except for peptide 9.

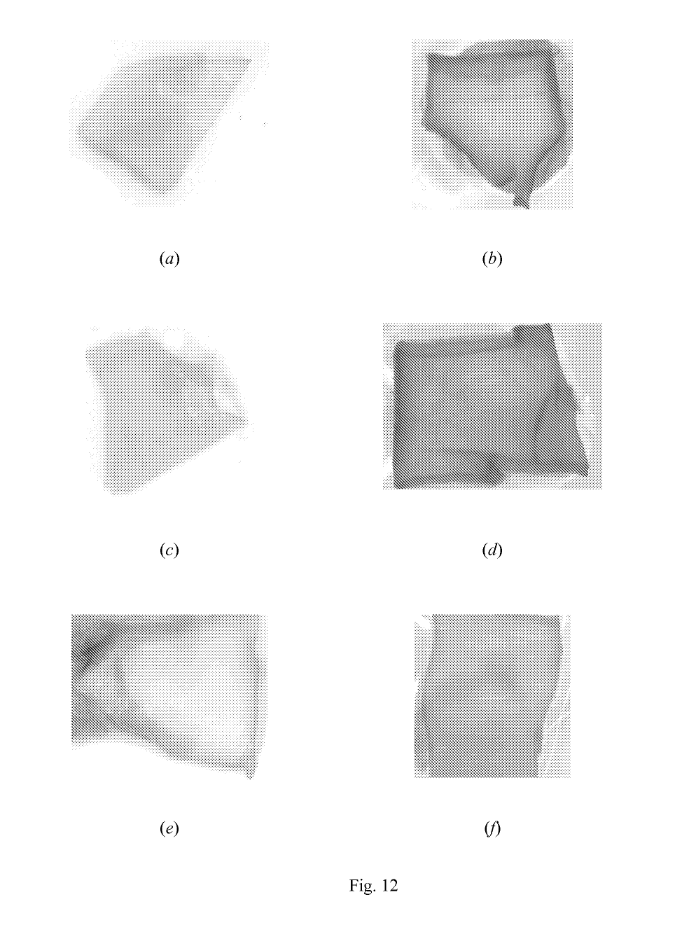

When peptide 9 was first assayed on NZW control tissue at the 1× concentration, it was seen to bind with a light, distinct staining pattern. Peptides 3 and 4 showed no binding to NZW tissue. These results are shown in FIG. 12. FIG. 12 shows the results of two separate experiments on two NZW rabbits. The images in (a), (c), and (e) show sections incubated with peptide 3, peptide 4, and the SA-HRP control, respectively and are from one experiment (exp199). The images in (b) and (d) show sections from a different experiment (exp214) incubated with peptide 9 (1× concentration), and the image in (f) shows the anti-FLAG control. Note that the sections incubated with peptide 9 in (b) and (d) show a subtle, yet distinct, brown staining pattern on the surface, while the control in (f) shows no staining. Therefore, peptide 9 initially showed targeting to the aortic surface on both the WHHL and NZW animal models. Peptides 3 and 4 show no staining on the NZW aorta samples as seen in images (a) and (c).

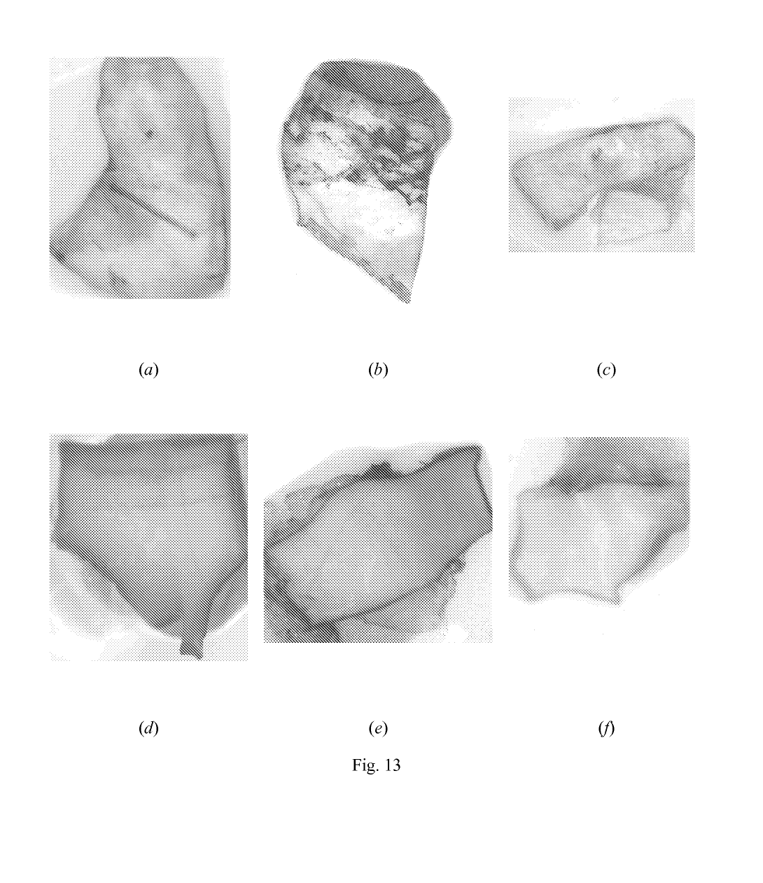

The fact that peptide 9 showed slight staining on the NZW tissue was originally interpreted to mean that peptide 9 binds the surface of rabbit aorta in a nonspecific manner. However, recent results show that when the concentration of peptide 9 is increased 10-fold (˜100 μg/mL), the staining shown on the WHHL tissue increases dramatically, while the staining on the NZW tissue remains the same as the staining seen with the 1× concentration. These results are shown below in FIG. 13. The top row shows the results on WHHL tissue while the bottom row is NZW tissue. The images in (a) and (d) show the staining caused by the 1× concentration of peptide 9 on WHHL and NZW tissue, respectively. The images in (b) and (e) show the staining of peptide 9 when the concentration is increased to the 10× concentration on WHHL and NZW tissue, respectively. Note the drastic difference in staining seen on the WHHL tissue when the concentration of peptide 9 is increased 10-fold from (a) to (b). However, there is little difference in the staining on the NZW tissue when the concentration of peptide 9 is increased by the same magnitude from (d) to (e). The control sections in (c) and (f) are from the 10× experiment and show no staining. These results indicate that peptide 9 targets plaque-rich WHHL tissue and that the staining on the NZW tissue is a consistent background staining.

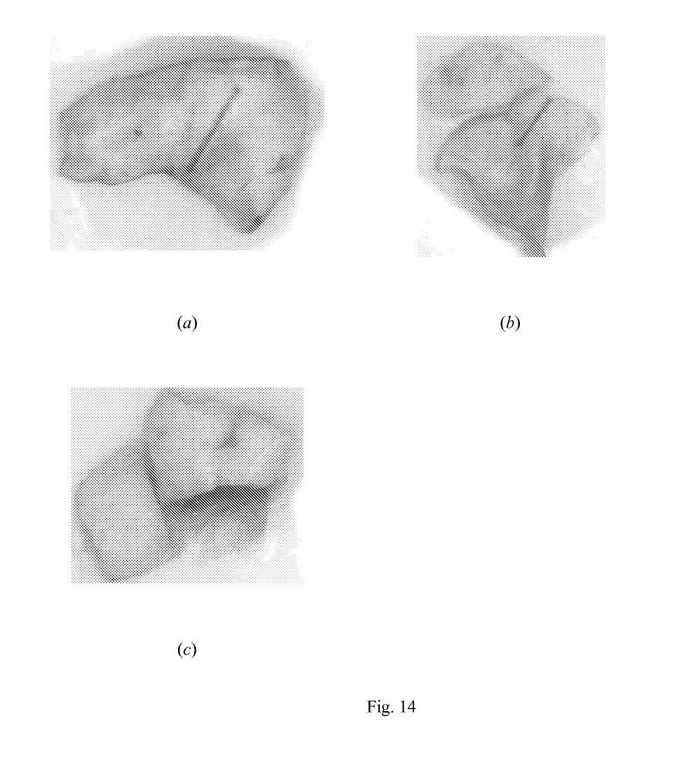

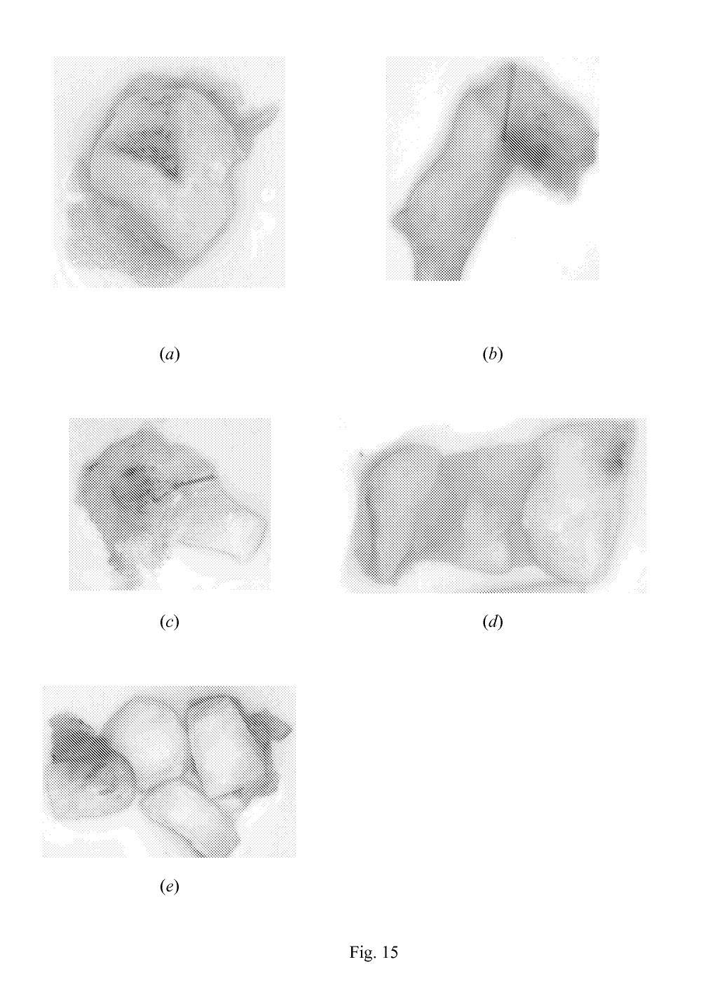

After the initial assays indicated that peptides 9, 3, and 4 were potential targeting candidates to WHHL rabbit aorta, peptides were synthesized that had randomly scrambled amino acid sequences corresponding to the original peptides. Peptide 9 (TQTPIKHHLLKE-FLAG) was randomly reordered to ILTHEHTLPKQ-FLAG, which will be referred to as peptide 9R (peptide 43 in Table 1). Similarly, peptides 3R and 4R were synthesized as Biotin-PPEEWSPVTHLL (peptide 44 in Table 1) and Biotin-RNPGYTYHIALG (peptide 45 in Table 1), respectively. The peptides were randomized such that no two amino acids in the original sequence appeared sequentially in the new (random) sequence. If the peptides are targeting plaque based on the order of the primary amino acid sequence, then the randomly sequenced peptides should show decreased binding activity. FIGS. 14 and 15 show the results of the binding assays comparing peptides 9, 3, and 4 to their scrambled counterparts, 9R, 3R, and 4R from one experiment on a WHHL rabbit.

FIG. 14 shows the results for peptides 9 and 9R, along with the corresponding control sections. The staining of peptide 9 in (a) is stronger than the staining seen from peptide 9R in (b), indicating that the primary amino acid sequence of peptide 9 is responsible for recognizing the surface of the aorta. Note that the control for this experiment (c) shows very slight staining, but it is much less than the staining present in (a). FIG. 15 shows the results for peptides 3 and 3R, 4 and 4R, and the corresponding control sections. Both randomized sequences in (b) and (d) show much less staining compared to the original sequences shown in (a) and (c), which is evidence that peptides 3 and 4 also bind due to the configuration of amino acids in the original sequences. Also, most of the control sections in (e) for peptides 3 and 4 show no staining

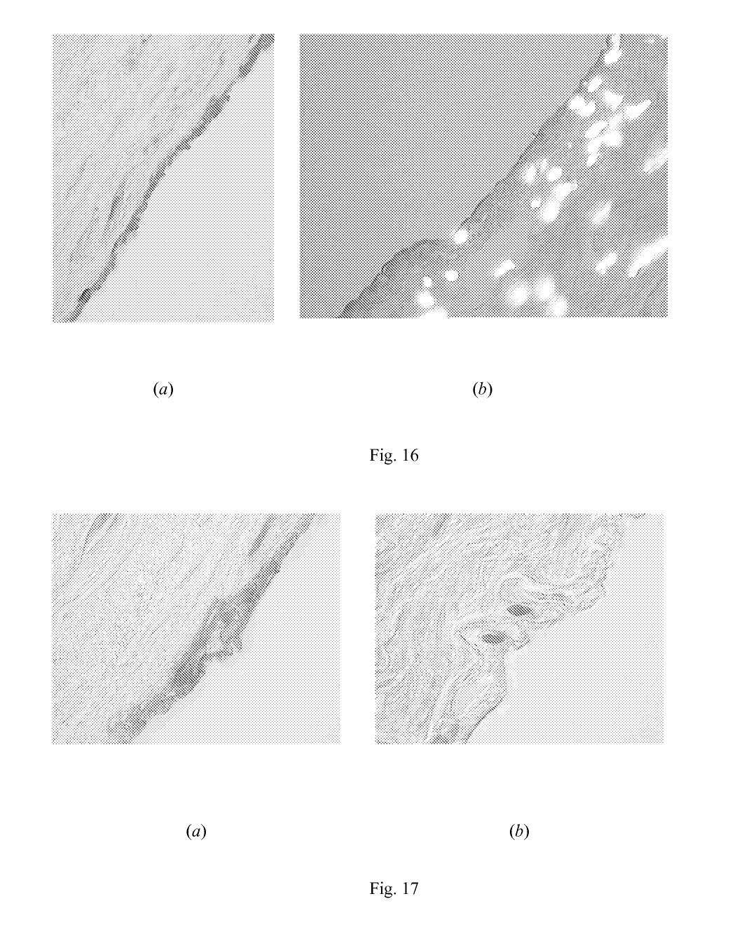

To demonstrate that the location of positive peptide binding is in fact the intimal layer of the aorta, several pieces of positively stained WHHL aorta with peptide 9 were sectioned for histological analysis. A Hoechst stain was performed on the tissue sections in order to visualize nuclei near the intima. The following figures show the results of this experiment. FIG. 16 shows a section of a plaque-laden region of WHHL tissue stained with peptide 9. The stain is clearly localized to the intimal layer, with no infiltration into the plaque region. Furthermore, the Hoechst stain in image (b) suggests an intact endothelial layer, but this should be confirmed with an endothelium-specific marker. Both images in FIG. 16 are 400× magnification.

It was also observed that most of the staining of peptide 9 is present on intima covering regions of plaque but not over non-plaque regions. This result is shown in FIG. 17. The image in (a) is taken over a plaque-rich region, while that in (b) is taken from a region with no observable plaque. A distinct difference in surface morphology distinguishes plaque from non-plaque regions. The intima covering non-plaque regions shows the characteristic wavy appearance seen in (b). Note that brown staining is only seen over the plaque-rich region in (a).

Staining was present on regions where the endothelium was removed. The endothelium could be removed by the detection assay or by the sectioning, and it is possible that the endothelium is degraded in vivo. Also, although most staining was present on intima covering plaque, some staining was seen on intima covering a small region that had no observable plaque, and staining was absent in a small area covering a plaque-rich region of the section. This indicates that the presence of plaque is neither a necessary nor sufficient condition for the binding of peptide 9 to the intimal aortic surface, which follows logically from the observation that peptide 9 shows slight background staining on NZW samples that have no observable plaque.

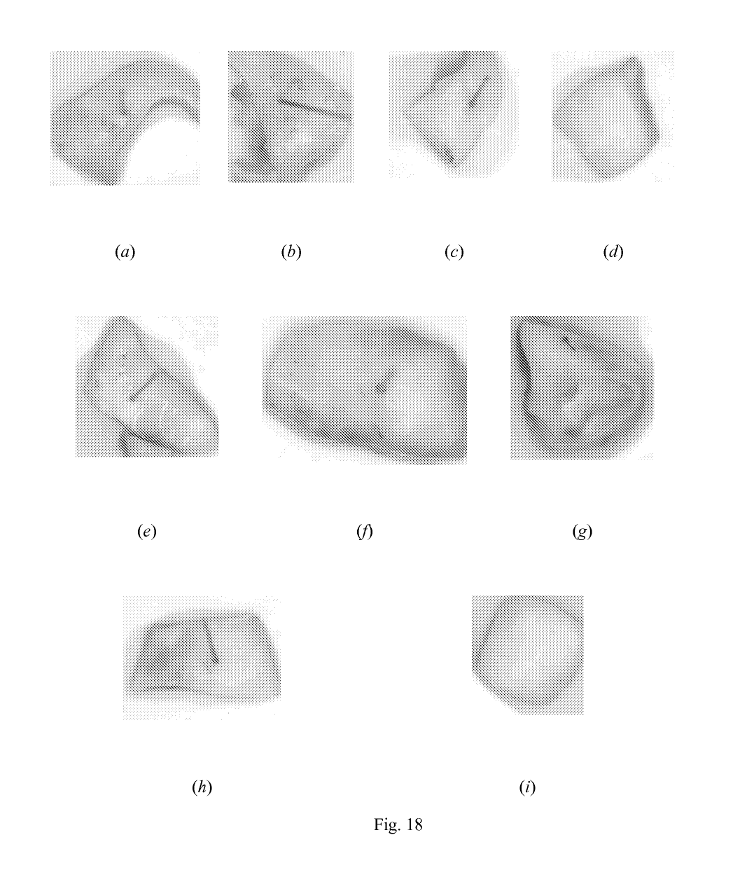

To demonstrate the variability in binding of the remaining peptides, the following figures (FIGS. 18-21) show staining of WHHL tissue samples from all of the peptides that showed any positive activity from four different experiments. Between five and seven tissue samples were incubated with all peptides in each experiment.

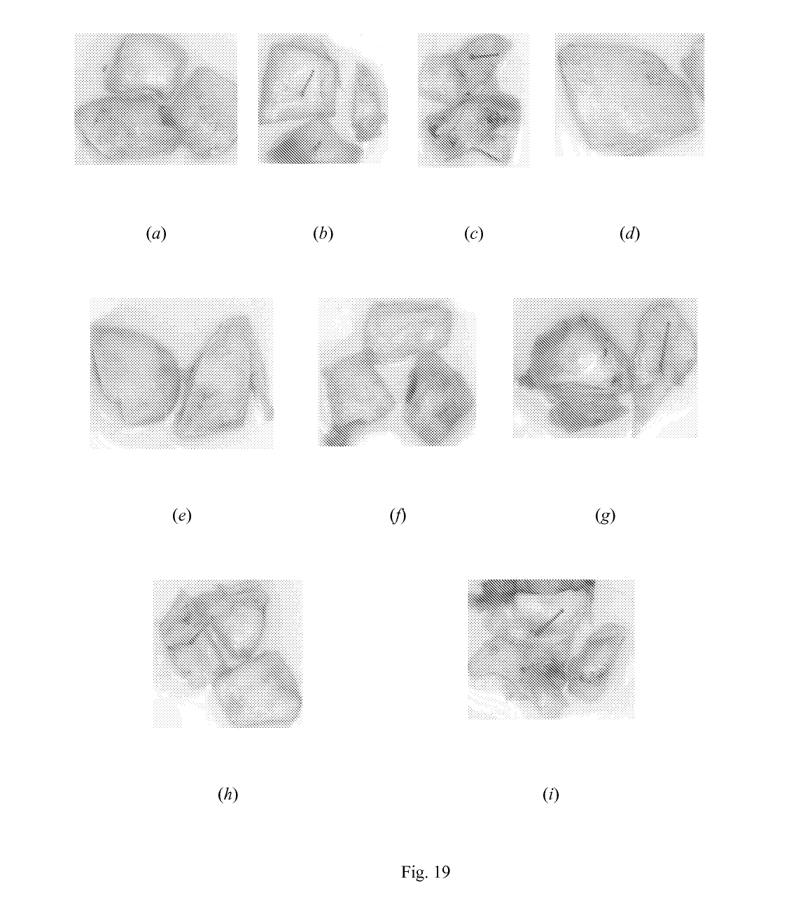

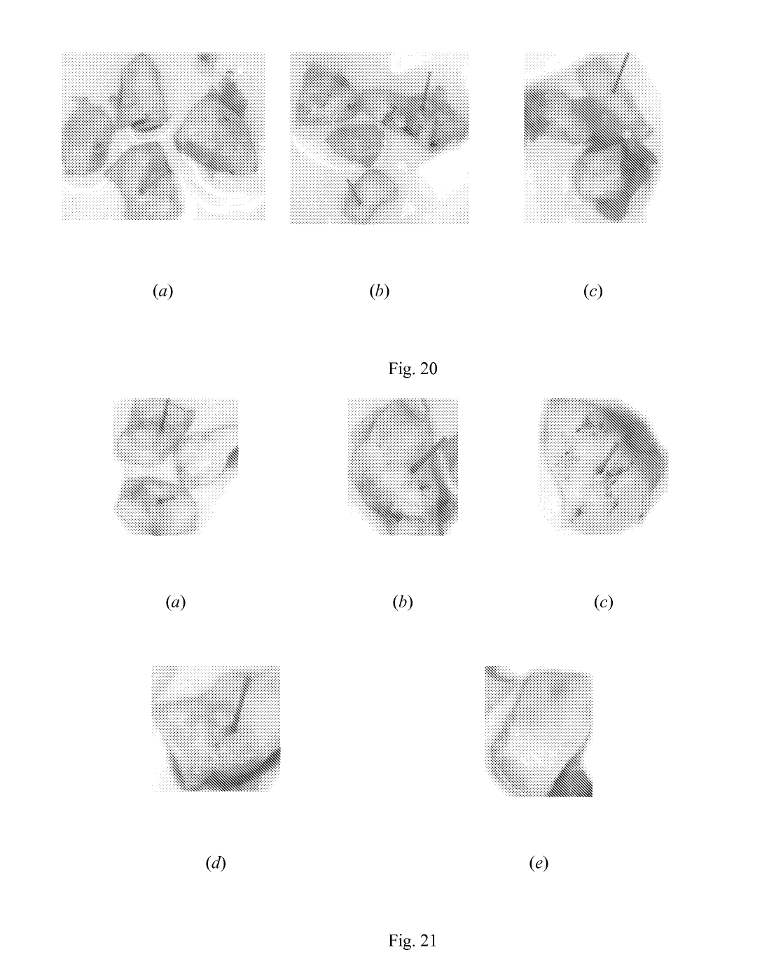

FIG. 18 shows the results. Peptides 1, 6, 7, 10, 11, 20, and 24 showed staining on one tissue sample. The controls for this experiment in (d) and (i) show no staining FIG. 19 shows tissue samples stained with peptides 2, 6, 7, 12, 13, 14, and 16 from exp217. The controls shown in (d) and (i) show minor staining. Note that this is the second observation of slight staining by peptides 6 and 7 on one of the several tissue samples that were incubated with peptide. FIGS. 20 and 21 demonstrate that peptides 24, 28, 34, 37, 38, and 39 all showed some staining on tissue samples from exp219 and exp223. The controls for these experiments in FIG. 20(c) and FIG. 21(e) show no staining. A summary of these results is shown in Table 2.

TABLE 2

Peptides that showed positive binding. Results are summarized for all
experiments, showing all peptides that displayed positive binding, excluding 9, 3, and 4.

| | | | |
|---|---|---|---|
| Exp201 | Exp217 | Exp219 | Exp223 |

| Peptide | | Peptide | | Peptide | | Peptide | |
|---|---|---|---|---|---|---|---|
| Number | Sequence | Number | Sequence | Number | Sequence | Number | Sequence |
| 1 | CKQSFEKSC-FLAG | 2 | CKSDANSHC-FLAG | 24 | Biotin- | 34 | Biotin- |
| | | | | | SSSKMQAAHQLP | | CHPASSPQC |
| 6 | Biotin-SSLPAPPWPLRG | 6 | Biotin-SSLPAPPWPLRG | 28 | Biotin- | 37 | Biotin- |
| | | | | | APHYLKTAPPPN | | TDTTMGQVHRHP |
| 7 | Biotin-TSPQTKDC | 7 | Biotin-TSPQTKDC | | | 38 | Biotin- |
| | | | | | | | NADNQMTWRHVL |
| 10 | CVMPGLKNC-FLAG | 12 | Biotin-CAPGPSKSC | | | 39 | Biotin- |
| | | | | | | | NLTSLTQGSAML |
| 11 | Biotin-CNHRYMQMC | 13 | Biotin-SIGYPLP | | | | |
| 20 | CNKNSIPHC-FLAG | 14 | CKQSPPSMC-FLAG | | | | |
| 24 | Biotin-SSSKMQAAHQLP | 16 | KSLSRHDHIHHH-FLAG | | | | |

### Example 4

### Frozen Tissue Assay Results

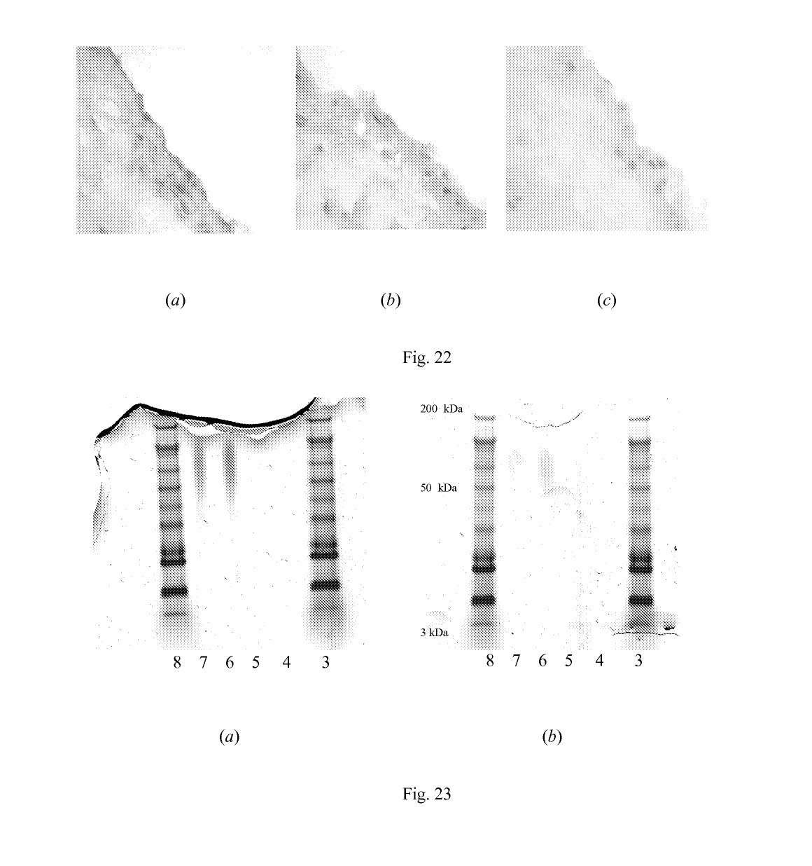

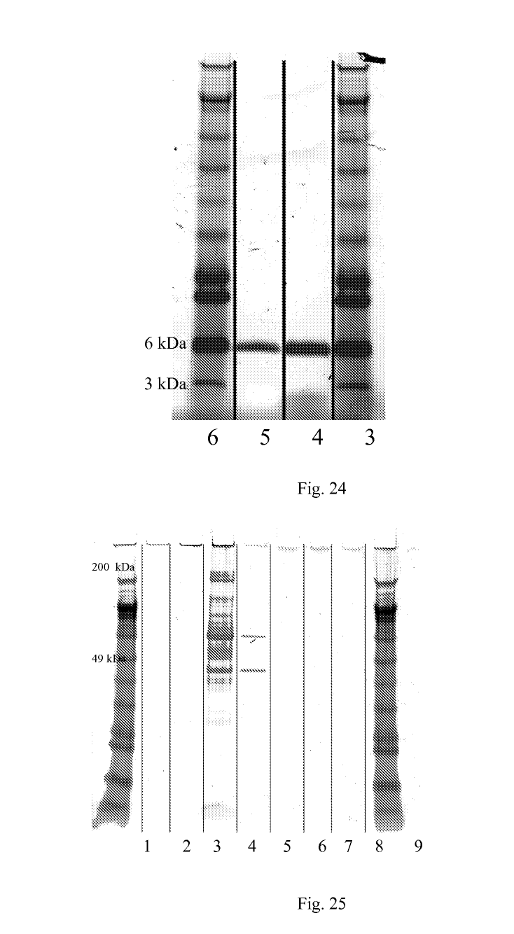

Several of the synthesized peptides were assayed for binding to frozen WHHL and NZW tissue as described above. Peptide 9 showed binding to the tissue samples. FIG. 22 shows the results of the binding assay for peptides 9 and 9R, and for the anti-FLAG-HRP control on WHHL tissue. The image shown in (a) is the result of incubating the tissue section with peptide 9. Note that the assay yields strong brown staining on the surface. The scrambled version of peptide 9, peptide 9R, was incubated with the tissue section shown in (b). There is no staining seen from peptide 9R, further supporting the hypothesis that the specific amino acid sequence of peptide 9 is responsible for binding. The anti-FLAG control is shown in (c). This also shows no brown staining. All images are 40× magnification.

### Example 5

### ApoE Knockout Mice Results

Peptides 7 and 9 have been tested for binding using in vivo circulation of peptide, followed by the removal of the aorta and assaying for activity as previously described. These experiments did not yield positive results. Peptides 7 and 9 have also been conjugated to iron oxide nanoparticles, injected into mice, and imaged in vivo using MRI to try to detect a change in signal between pre- and post-contrast particle injection.

The present invention should not be considered limited to the specific examples described above, but rather should be understood to cover all aspects of the invention. Various modifications, equivalent processes, as well as numerous structures and devices to which the present invention may be applicable will be readily apparent to those of skill in the art.

We claim:

1. A targeting polypeptide imaging agent comprising at least one targeting polypeptide in spatial proximity to at least one contrast enhancing imaging agent, wherein the targeting polypeptide has a sequence selected from:
C-K-Q-S-F-E-K-S-C;
S-S-L-P-A-P-P-W-P-L-R-G;
T-S-P-Q-T-K-D-C;
C-V-M-P-G-L-K-N-C;
C-N-H-R-Y-M-Q-M-C;
C-N-K-N-S-I-P-H-C;
S-S-S-K-M-Q-A-A-H-Q-L-P;
C-K-S-D-A-N-S-H-C;
C-A-P-G-P-S-K-S-C;
S-I-G-Y-P-L-P;
C-K-Q-S-P-P-S-M-C;
K-S-L-S-R-H-D-H-I-H-H-H;
A-P-H-Y-L-K-T-A-P-P-P-N;
C-H-P-A-S-S-P-Q-C;
T-D-T-T-M-G-Q-V-H-R-H-P;
N-A-D-N-Q-M-T-W-R-H-V-L;
N-L-T-S-L-T-Q-G-S-A-M-L;
T-P-L-E-V-H-P-L-S-L-P-W;
Y-I-T-P-Y-A-H-L-R-G-G-N;
and
T-Q-T-P-I-K-H-H-L-L-K-E,
or
a sequence having at least 50% identity to one of the targeting polypeptide sequences.

2. The targeting polypeptide imaging agent of claim 1, wherein the at least one targeting polypeptide is covalently or non-covalently chemically bonded to the at least one contrast enhancing imaging agent.

3. The targeting polypeptide imaging agent of claim 1, wherein the targeting polypeptide has a sequence of T-P-L-E-V-H-P-E-S-L-P-W.

4. The targeting polypeptide imaging agent of claim 1, wherein the targeting polypeptide has a sequence of Y-I-T-P-Y-A-H-L-R-G-G-N.

5. The targeting polypeptide imaging agent of claim 1, wherein the targeting polypeptide has a sequence of T-Q-T-P-I-K-H-H-L-L-K-E.

6. The targeting polypeptide imaging agent of claim 1, wherein the at least one contrast enhancing imaging agent is a paramagnetic contrast agent.

7. The targeting polypeptide imaging agent of claim 6, wherein the paramagnetic contrast agent is selected from as gadolinium, cobalt, nickel, manganese, small particulate iron oxides, and ultra small particulate iron oxides, or combinations thereof.

8. The targeting polypeptide imaging agent of claim 1, wherein the at least one contrast enhancing imaging agent comprises a contrast enhancing imaging agent selected from ¹³¹I, ¹²⁵I, ¹²³I, ^{99m}Tc, ¹⁸F, ⁶⁸Ga, ⁶⁷Ga, ⁷²As, ⁸⁹Zr, ⁶⁴Cu, ⁶²Cu, ¹¹¹In, ²⁰³Pb, ¹⁹⁸Hg, ¹¹C, ⁹⁷Ru, and ²⁰¹TI, or combinations thereof.

9. The targeting polypeptide imaging agent of claim 1, wherein the at least one contrast enhancing imaging agent comprises a contrast-enhancing nanodevice.

10. The targeting polypeptide imaging agent of claim 1, wherein the targeting polypeptide sequence further comprises a C-terminal sequence comprising D-Y-K-D-D-D-K.

11. The targeting polypeptide imaging agent of claim 1, wherein the targeting polypeptide sequence further comprises at least one glycine linker at the N-terminal or C-terminal.

12. The targeting polypeptide imaging agent of claim 1, wherein the targeting polypeptide sequence is N-terminal biotinylated.

13. A method of detecting vulnerable plaque comprising the steps of:
supplying at least one targeting polypeptide imaging agent comprising at least one targeting polypeptide in spatial proximity to at least one contrast enhancing imaging agent such that the at least one targeting polypeptide imaging agent may come in contact with a target of interest; and subsequently applying an imaging technique to image the at least one targeting polypeptide imaging agent, wherein the targeting polypeptide has a sequence selected from;
C-K-Q-S-F-E-K-S-C;
S-S-L-P-A-P-P-W-P-L-R-G;
T-S-P-Q-T-K-D-C;
C-V-M-P-G-L-K-N-C;
C-N-H-R-Y-M-Q-M-C;
C-N-K-N-S-I-P-H-C;
S-S-S-K-M-Q-A-A-H-Q-L-P;
C-K-S-D-A-N-S-H-C;
C-A-P-G-P-S-K-S-C;
S-I-G-Y-P-L-P;
C-K-Q-S-P-P-S-M-C;
K-S-L-S-R-H-D-H-I-H-H-H;
A-P-H-Y-L-K-T-A-P-P-P-N;
C-H-P-A-S-S-P-Q-C;
T-D-T-T-M-G-Q-V-H-R-H-P;
N-A-D-N-Q-M-T-W-R-H-V-L;
N-L-T-S-L-T-Q-G-S-A-M-L;
T-P-L-E-V-H-P-E-S-L-P-W;
Y-I-T-P-Y-A-H-L-R-G-G-N;
and
T-Q-T-P-I-K-H-H-L-L-K-E,
or
a sequence having at least 50% identity to one of the polypeptide sequences.

14. The method of claim 13, wherein the step of supplying at least one targeting polypeptide imaging agent comprises supplying the at least one targeting polypeptide imaging agent in vitro.

15. The method of claim 13, wherein the step of supplying at least one targeting polypeptide imaging agent comprises supplying the at least one targeting polypeptide imaging agent in vivo to a subject, and wherein the step of subsequently applying an imaging technique comprises applying an imaging technique to the subject.

16. The method of claim 15 wherein the step of supplying at least one targeting polypeptide imaging agent comprises injecting a subject with the at least one targeting polypeptide imaging agent.

17. The method of claim 15 wherein the subject is a human subject.

18. The method of claim 13, wherein the polypeptide has a sequence of T-P-L-E-V-H-P-E-S-L-P-W.

19. The method of claim 13, wherein the polypeptide has a sequence of Y-I-T-P-Y-A-H-L-R-G-G-N.

20. The method of claim 13, wherein the polypeptide has a sequence of T-Q-T-P-I-K-H-H-L-L-K-E.

21. The method of claim 13, wherein the imaging technique is selected from computed tomography scanning, computerized axial tomography scanning, positron emission tomography scanning, gamma detection, and magnetic resonance imaging or combinations thereof.

22. A kit, comprising at least one targeting polypeptide imaging agent in combination with a pharmaceutically acceptable carrier, wherein the at least one targeting polypeptide imaging agent comprises at least one targeting polypeptide in spatial proximity to at least one contrast enhancing imaging agent, and wherein the targeting polypeptide has a sequence selected from:
C-K-Q-S-F-E-K-S-C;
S-S-L-P-A-P-P-W-P-L-R-G;
T-S-P-Q-T-K-D-C;
C-V-M-P-G-L-K-N-C;
C-N-H-R-Y-M-Q-M-C;
C-N-K-N-S-I-P-H-C;
S-S-S-K-M-Q-A-A-H-Q-L-P;
C-K-S-D-A-N-S-H-C;
C-A-P-G-P-S-K-S-C;
S-I-G-Y-P-L-P;
C-K-Q-S-P-P-S-M-C;
K-S-L-S-R-H-D-H-I-H-H-H;
A-P-H-Y-L-K-T-A-P-P-P-N;
C-H-P-A-S-S-P-Q-C;
T-D-T-T-M-G-Q-V-H-R-H-P;
N-A-D-N-Q-M-T-W-R-H-V-L;
N-L-T-S-L-T-Q-G-S-A-M-L;
T-P-L-E-V-H-P-E-S-L-P-W;
Y-I-T-P-Y-A-H-L-R-G-G-N;
and
T-Q-T-P-I-K-H-H-L-L-K-E,
or a sequence having at least 50% identity to one of the targeting polypeptide sequences.
